(19) United States
(12) Patent Application Publication
(10) Pub. No.: US 2019/0154708 A1
(43) Pub. Date: May 23, 2019

(54) BIOMARKERS FOR PREDICTING DEGREE OF WEIGHT LOSS

(71) Applicant: NESTEC S.A., Vevey (CH)

(72) Inventors: Irina Irincheeva, Cossonay-Ville (CH); Jorg Hager, Houtaud (FR); Loic Dayon, Colombier (CH); Ornella Cominetti, Preverenges (CH)

(21) Appl. No.: 16/091,390

(22) PCT Filed: Apr. 6, 2017
(86) PCT No.: PCT/EP2017/058183

§ 371 Date: Oct. 4, 2018

(30) Foreign Application Priority Data
Apr. 6, 2016 (EP) 16164025.5

(51) Int. Cl. G01N 33/86

(52) U.S. Cl. CPC G01N 33/86; G01N 2333/96461

(57) ABSTRACT

Biomarkers for predicting weight loss The present invention provides a method for predicting the degree of weight loss attainable by applying one or more dietary interventions to a subject, said method comprising; determining the level of one or more biomarkers in one or more samples obtained from the subject, wherein the one or more biomarkers include vitamin K-dependent protein C.

### FIELD OF INVENTION

The present invention provides a number of biomarkers and biomarker combinations that can be used to predict the degree of weight loss attainable by applying one or more dietary interventions to a subject.

### BACKGROUND

Obesity is a chronic metabolic disorder that has reached epidemic proportions in many areas of the world. Obesity is the major risk factor for serious co-morbidities such as type 2 diabetes mellitus, cardiovascular disease, dyslipidaemia and certain types of cancer (World Health Organ Tech Rep Ser. 2000; 894:i-xii, 1-253).

It has long been recognized that low calorie dietary interventions can be very efficient in reducing weight and that this weight loss is generally accompanied by an improvement for the risk of obesity related co-morbidities, in particular type 2 diabetes mellitus (World Health Organ Tech Rep Ser. 2000; 894:i-xii, 1-253). Empirical data suggests that a weight loss of at least 10% of the initial weight results in a considerable decrease in risk for obesity related co-morbidities (World Health Organ Tech Rep Ser. 2000; 894:i-xii, 1-253). However, the capacity to lose weight shows large inter-subject variability.

Some studies (e.g. Ghosh, S. et al., Obesity (Silver Spring), (2011) 19(2):457-463) illustrate that a percentage of the population do not successfully lose weight on a low calorie diet. This leads to an unrealistic expectation of weight loss, which in turn causes non-compliance, drop-outs and generally unsuccessful dietary intervention.

Some studies also demonstrate that there are methods in the art for monitoring weight loss which include monitoring levels of particular biomarkers in plasma (e.g. Lijnen et al., Thromb Res. 2012 January, 129(1): 74-9; Cugno et al., Intern Emerg Med. 2012 June, 7(3): 237-42; and Bladbjerg et al., Br J Nutr. 2010 December, 104(12): 1824-30). However, these methods do not provide a prediction or indication of the degree of weight loss attainable by a particular subject. There is no predictive value in looking at the correlation of biomarker levels with weight loss.

The solution for successful planning and design of dietary interventions, for example low calorie diets, lies in the availability of a method which predicts a weight loss trajectory. Such a method would be useful to assist in modifying a subject's lifestyle, e.g. by a change in diet, and also to stratify subjects into adapted treatment groups according to their biological weight loss capacity.

United States Patent Application US 2011/0124121 discloses a method for predicting weight loss success. The methods disclosed comprises selecting a patient who is undergoing or considering undergoing a weight loss therapy such as gastric banding, measuring one or more hormone responses of the patient to caloric intake and predicting success of a weight loss therapy based on the hormone response. The hormones measured are gastrointestinal hormones such as a pancreatic hormone.

European Patent Application EP 2 420 843 discloses a method for determining the probability that a person will maintain weight loss after an intentional weight loss by determining the level of angiotensin I converting enzyme (ACE) before and after the dietary period.

There is, however, still a need for a method of accurately predicting the degree of weight loss in a subject. Consequently, it was the objective of the present invention to provide biomarkers that can be detected easily and that can facilitate the prediction of weight loss in a subject. Such biomarkers can be used to predict weight trajectory of a subject prior to a dietary intervention. These biomarkers can be used to optimise dietary intervention and assist in lifestyle modifications.

### SUMMARY OF THE INVENTION

The present invention investigates the level of one or more biomarkers in order to predict the degree of weight loss attainable by applying one or more dietary interventions to a subject. In particular, the inventors have found that certain biomarkers can be used to reliably predict the weight loss attainable by a subject following a low calorie diet.

Accordingly in one aspect the present invention provides a method for predicting the degree of weight loss attainable by applying one or more dietary interventions to a subject, said method comprising determining the level of one or more biomarkers in one or more samples obtained from the subject, wherein the one or more biomarkers include vitamin K-dependent protein C.

In one embodiment, the method further comprises determining the level of one or more biomarkers selected from coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In one embodiment the method further comprises determining the level of each of coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In one embodiment the method comprises determining the level of each of vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34 and interleukin-17 receptor C in one or more samples.

The level of the one or more biomarkers may be compared to a reference value, wherein the comparison is indicative of the predicted degree of weight loss attainable by the subject. The reference value may be based on a value (e.g. an average) of the one or more biomarkers in a population of subjects who have previously undergone the dietary intervention.

In one embodiment, the levels of each of vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 are determined, and increased levels of vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; protein Z-dependent protease inhibitor; serum paraoxonase/arylesterase 1; complement factor D; carbonic anhydrase 6; and angiopoietin-1 and decreased levels of leukocyte immunoglobulin-like receptor subfamily B member 2; X-ray repair cross-complementing protein 6; caspase-2; interleukin-34; interleukin-17 receptor C; plasminogen; leptin and macrophage metalloelastase in the sample is indicative of a greater degree of weight loss in the subject.

In one embodiment, the one or more samples are derived from blood, e.g. a blood plasma sample.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include coagulation factor XIII A chain. The method may further comprise determining the level of one or more biomarkers selected vitamin K-dependent protein C; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include pigment epithelium-derived factor. The method may further comprise determining the level of one or more biomarkers selected vitamin K-dependent protein C; coagulation factor XIII A chain; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include serum amyloid P-component. The method may further comprise determining the level of one or more biomarkers selected vitamin K-dependent protein C; coagulation factor XIII A chain; pigment epithelium-derived factor; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include neuroligin-4, X-linked. The method may further comprise determining the level of one or more biomarkers selected vitamin K-dependent protein C; coagulation factor XIII A chain; pigment epithelium-derived factor; serum amyloid P-component; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include CD226 antigen. The method may further comprise determining the level of one or more biomarkers selected vitamin K-dependent protein C; coagulation factor XIII A chain; pigment epithelium-derived factor; serum amyloid P-component; neuroligin-4, X-linked; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include metalloproteinase inhibitor 3. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include eukaryotic translation initiation factor 4E-binding protein 2 is determined. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include leukocyte immunoglobulin-like receptor subfamily B member 2 is determined. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include X-ray repair cross-complementing protein 6. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include caspase-2. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include interleukin-34. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include interleukin-17 receptor C. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include protein Z-dependent protease inhibitor. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; serum paraoxonase/arylesterase 1; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include serum paraoxonase/arylesterase 1. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; plasminogen; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include plasminogen. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; complement factor D; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include complement factor D. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; leptin; carbonic anhydrase 6; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include leptin. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; Interleukin-34; Interleukin-17 receptor C; protein Z-dependent protease inhibitor; serum paraoxonase/arylesterase 1; Plasminogen; Complement factor D; Carbonic anhydrase 6; Macrophage metalloelastase and Angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include carbonic anhydrase 6. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; macrophage metalloelastase and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include macrophage metalloelastase. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6 and angiopoietin-1 in one or more samples.

In another aspect the present invention provides a method for predicting the degree of weight loss attainable by applying one or more dietary interventions to a subject, said method comprising determining the level of one or more biomarkers in one or more samples obtained from the subject, wherein the one or more biomarkers include angiopoietin-1. The method may further comprise determining the level of one or more biomarkers selected vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; Plasminogen; complement factor D; leptin; carbonic anhydrase 6 and macrophage metalloelastase in one or more samples.

Preferably the dietary intervention is a low calorie diet. In one embodiment, the low calorie diet comprises a calorie intake of about 600 to about 1200 kcal/day. The low calorie diet may comprise administration of at least one diet product. Preferably the diet product is Optifast® or Modifast®. The low calorie diet may also comprise administration of up to, for example, about 400 g vegetables/day.

In one embodiment, the diet may comprise a product such as Optifast® or Modifast®. This may be supplemented with three portions of non-starchy vegetables such that the total energy intake is about 2.5 MJ (600 kcal/day). This may be further supplemented with at least 2 L of water or other energy free beverages per day.

In another embodiment, the diet may comprise, for example, a composition which is 46.4% carbohydrate, 32.5% protein and 20.1% with fat, vitamins, minerals and trace elements; 2.1MJ per day (510 kcal/day); This may be supplemented with three portions of non-starchy vegetables such that the total energy intake is about 2.5 MJ (600 kcal/day). This may be further supplemented with at least 2 L of water or other energy free beverages per day.

In one embodiment, the low calorie diet has a duration of up to 12 weeks, e.g. 6 to 12 weeks.

In one embodiment, the method further comprises combining the level of the one or more biomarkers with one or more anthropometric measures and/or lifestyle characteristics of the subject. Preferably the anthropometric measure is selected from the group consisting of gender, weight, height, age and body mass index, and the lifestyle characteristic is whether the subject is a smoker or a non-smoker.

In one embodiment, the degree of weight loss is represented by the body mass index (BMI) that a subject is predicted to attain by applying the dietary intervention. This may be termed BMI2 and be calculated using formula (1):

BMI2=c1*BMI1+c2*age−c3*vitamin K-dependent protein C−c4*coagulation factor XIII A chain−c5*pigment epithelium-derived factor−c6*Serum amyloid P-component−c7*Neuroligin-4,X-linked-c8*CD226antigen−c9*Metalloproteinase inhibitor 3−c10*Eukaryotic translation initiation factor 4E-binding protein 2+c11*Leukocyte immunoglobulin-like receptor subfamily B member 2+c12*X-ray repair cross-complementing protein 6+c13*Caspase-2+c14*Interleukin-34+c15*Interleukin-17 receptor C−c16*Protein Z-dependent protease inhibitor−c17*Serum paraoxonase/arylesterase 1+c18*Plasminogen−c19*Complement factor D+c20*Leptin−c21*Carbonic anhydrase 6+c22*Macrophage metalloelastase−c23*Angiopoietin-1; (1)

wherein BMI1 is the subject's body mass index before the dietary intervention and BMI2 is the subject's predicted body mass index after the dietary intervention; and wherein c1 to c23 are positive integers.

According to a further aspect, the present invention provides a method for optimizing one or more dietary interventions for a subject comprising predicting the degree of weight loss attainable by the subject according to a method as defined herein, and applying the dietary intervention to the subject.

In a further aspect, the present invention provides a method for predicting the body mass index that a subject would be expected to attain from a dietary intervention (BMI2), wherein the method comprises determining the level of one or more biomarkers as defined herein in one or more samples obtained from the subject, and predicting BMI2 using formula (1) as described herein.

In a further aspect of the present invention there is provided a method for selecting a modification of lifestyle of a subject, the method comprising (a) performing a method as defined herein, and (b) selecting a suitable modification in lifestyle based upon the degree of weight loss predicted.

In one embodiment, the modification of lifestyle comprises a dietary intervention. The dietary intervention may comprise administering at least one diet product to the subject. For example, the dietary intervention may be a low calorie diet. A low calorie diet may comprise a decreased consumption of fat and/or an increase in consumption of low fat foods. By way of example only, low fat foods may include wholemeal flour and bread, porridge oats, high-fibre breakfast cereals, wholegrain rice and pasta, vegetables and fruit, dried beans and lentils, baked potatoes, dried fruit, walnuts, white fish, herring, mackerel, sardines, kippers, pilchards, salmon and lean white meat.

In a further aspect of the present invention there is provided a diet product for use as part of a low calorie diet for weight loss, wherein the diet product is administered to a subject that is predicted to attain a degree of weight loss by the methods described herein.

In one aspect, the diet product may comprise a product such as Optifast® or Modifast®. This may be supplemented with three portions of non-starchy vegetables such that the total energy intake is about 2.5 MJ (600 kcal/day). This may be further supplemented with at least 2 L of water or other energy free beverages per day.

In another aspect, the diet product may comprise, for example, a composition which is 46.4% carbohydrate, 32.5% protein and 20.1% with fat, vitamins, minerals and trace elements; 2.1MJ per day (510 kcal/day); This may be supplemented with three portions of non-starchy vegetables such that the total energy intake is about 2.5 MJ (600 kcal/day). This may be further supplemented with at least 2 L of water or other energy free beverages per day.

In a further aspect of the present invention there is provided a diet product for use in treating obesity or an obesity-related disorder, wherein the diet product is administered to a subject that is predicted to attain a degree of weight loss by the methods defined herein.

In a further aspect of the present invention, there is provided the use of a diet product in a low calorie diet for weight loss wherein the diet product is administered to a subject that is predicted to attain a degree of weight loss by the methods defined herein.

In a further aspect of the present invention, there is provided a computer program product comprising computer implementable instructions for causing a programmable computer to predict the degree of weight loss attainable by a subject according to the methods described herein.

In a further aspect of the present invention, there is provided a computer program product comprising computer implementable instructions for causing a programmable computer to predict the degree of weight loss given the levels of one or more biomarkers from the user, wherein the biomarkers are selected from one or more biomarkers as described herein.

In a further aspect of the present invention, there is provided a kit for predicting the degree of weight loss attainable by a subject following a dietary intervention, wherein said kit comprises at least two antibodies, wherein each antibody is specific for a biomarker as described herein.

### DETAILED DESCRIPTION OF THE INVENTION

### Predicting the Degree of Weight Loss

The present invention relates in one aspect to a method of predicting the degree of weight loss attainable by applying one or more dietary interventions to a subject. In particular embodiments, the method may be used to make an informed prediction of the subject's capacity to lose weight, and select or adjust one or more dietary intervention accordingly. For example, where the dietary intervention is a low calorie diet, the method could be used to select the appropriate diet for the subject or to adjust the daily calorie intake or duration of a particular diet to affect the degree of weight loss, or to increase compliance to the low calorie diet by setting realistic expectations for the subject. The method may also be used to assist in modifying the lifestyle of a subject.

The method provides a skilled person with a useful tool for assessing which subjects will most likely benefit from a particular dietary intervention, e.g. a low calorie diet. The present method therefore enables dietary interventions such as a low calorie diet and modifications in lifestyle to be optimised.

Weight loss as defined herein may refer to a reduction in parameters such as weight (e.g. in kilograms), body mass index (kgm⁻²), or waist circumference (e.g. in centimetres), or waist-hip ratio (e.g. in centimetres). Weight loss may be calculated by subtracting the value of one or more of the aforementioned parameters at the end of the dietary intervention from the value of said parameter at the onset of the dietary intervention. Preferably, the degree of weight loss is represented by the body mass index that a subject is predicted to attain by applying the dietary intervention.

The degree of weight loss may be expressed as a percentage of a subject's body weight (e.g. in kilograms) or body mass index (kgm⁻²). For example, a subject may be predicted to lose at least 10% of their initial body weight, at least 8% of their initial body weight, or at least 5% of their initial body weight. By way of example only, a subject may be predicted to lose between 5 and 10% of their initial body weight.

In one embodiment, the percentage may be associated with an obesity-related disorder. For example, a degree of weight loss of at least 10% of initial body weight results in a considerable decrease in risk for obesity related co-morbidities.

Based on the degree of weight loss predicted using the methods defined herein, subjects may be stratified into one or more groups or categories. For example, subjects may be stratified according to whether or not they are predicted to lose a significant amount of weight.

### Subject

Preferably the subject is a mammal, preferably a human. The subject may alternatively be a non-human mammal, including for example, a horse, cow, sheep or pig. In one embodiment, the subject is a companion animal such as a dog or a cat.

### Sample

The present invention comprises a step of determining the level of one or more biomarkers in one or more samples obtained from a subject.

Preferably the sample is derived from blood or urine. More preferably the sample is derived from blood. The sample may contain a blood fraction or may be wholly blood. The sample preferably comprises blood plasma or serum, most preferably blood plasma. Techniques for collecting samples from a subject are well known in the art.

### Dietary Intervention

By the term “dietary intervention” is meant an external factor applied to a subject which causes a change in the subject's diet. In one embodiment, the dietary intervention is a low calorie diet.

Preferably the low calorie diet comprises a calorie intake of about 600 to about 1500 kcal/day, more preferably about 600 to about 1200 kcal/day, most preferably about 800 kcal/day. In one embodiment, the low calorie diet may comprise a predetermined amount (in grams) of vegetables per day, preferably up to about 400 g vegetables/day, e.g. about 200 g vegetables/day.

The low calorie diet may comprise administration of at least one diet product. The diet product may be a meal replacement product or a supplement product which may e.g. suppress the subject's appetite. The diet product can include food products, drinks, pet food products, food supplements, nutraceuticals, food additives or nutritional formulas.

In one embodiment, the diet may comprise a product such as Optifast® or Modifast®. This may be supplemented with three portions of non-starchy vegetables such that the total energy intake is about 2.5 MJ (600 kcal/day). This may be further supplemented with at least 2 L of water or other energy free beverages per day.

In another embodiment, the diet may comprise, for example, a composition which is 46.4% carbohydrate, 32.5% protein and 20.1% with fat, vitamins, minerals and trace elements; 2.1MJ per day (510 kcal/day); This may be supplemented with three portions of non-starchy vegetables such that the total energy intake is about 2.5 MJ (600 kcal/day). This may be further supplemented with at least 2 L of water or other energy free beverages per day.

In one embodiment, the low calorie diet has a duration of up to 12 weeks. Preferably the low calorie diet has a duration of between 6 and 12 weeks, preferably between 8 and 10 weeks, e.g. 8 weeks.

### Determining the Level of One or More Biomarkers in the Sample

In one embodiment, the level of one or more biomarkers is determined prior to the dietary intervention. In another embodiment, the level of one or more biomarkers is determined prior to, and after the dietary intervention. The biomarker level may also be determined at predetermined times throughout the dietary intervention. These predetermined times may be periodic throughout the dietary intervention, e.g. every day or three days, or may depend on the subject being tested, the type of sample being analysed and/or the degree of weight loss which is predicted to be attained.

When obtained prior to the dietary intervention, the biomarker level may be termed the “fasting level”. When obtained after the dietary intervention, the biomarker level may be termed the “calorie intake level”. For example, the biomarker level may be determined at fasting, or at fasting and after calorie intake. Most preferably the fasting level of each biomarker is determined.

The level of the individual biomarker species in the sample may be measured or determined by any suitable method known in the art. For example, mass spectrometry (MS), antibody detection methods, e.g. enzyme-linked immunoabsorbent assay (ELISA), non-antibody protein scaffolds (e.g. fibronectin scaffolds), radioimmuno-assay (RIA), or aptamers may be used. Other spectroscopic methods, chromatographic methods, labelling techniques, or quantitative chemical methods may also be used.

In one embodiment, the level of one or more biomarkers may be determined by staining the sample with a reagent that labels one or more of the biomarkers. “Staining” is typically a histological method which renders the biomarker detectable by microscopic techniques such as those using visible or fluorescent light. Preferably the biomarker is detected in the sample by immunohistochemistry (IHC). In IHC, the biomarker may be detected by an antibody which binds specifically to one or more of the biomarkers. Suitable antibodies are known or may be generated using known techniques. Suitable test methods for detecting antibody levels include, but are not limited to, an immunoassay such as an enzyme-linked immunosorbent assay, radioimmunoassay, Western blotting and immunoprecipitation.

The antibody may be a monoclonal antibody, polyclonal antibody, multispecific antibody (e.g., bispecific antibody), or fragment thereof provided that it specifically binds to the biomarker being detected. Antibodies may be obtained by standard techniques comprising immunizing an animal with a target antigen and isolating the antibody from serum. Monoclonal antibodies may be made by the hybridoma method first described by Kohler et al., Nature 256:495 (1975), or may be made by recombinant DNA methods (see, e.g., U.S. Pat. No. 4,816,567). The monoclonal antibodies may also be isolated from phage antibody libraries using the techniques described in Clackson et al., Nature 352:624-628 (1991) and Marks et al., J. Mol. Biol. 222:581-597 (1991), for example. The antibody may also be a chimeric or humanized antibody. Antibodies are discussed further below.

Two general methods of IHC are available; direct and indirect assays. According to the first assay, binding of antibody to the target antigen is determined directly. This direct assay uses a labelled reagent, such as a fluorescent tag or an enzyme-labelled primary antibody, which can be visualized without further antibody interaction.

In a typical indirect assay, unconjugated primary antibody binds to the antigen and then a labelled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC may be labelled with a detectable moiety. Numerous labels are available, including radioisotopes, colloidal gold particles, fluorescent labels and various enzyme-substrate labels. Fluorescent labels include, but are not limited to, rare earth chelates (europium chelates), Texas Red, rhodamine, fluorescein, dansyl, Lissamine, umbelliferone, phycocrytherin and phycocyanin, and/or derivatives of any one or more of the above. The fluorescent labels can be conjugated to the antibody using known techniques.

Various enzyme-substrate labels are available, e.g. as disclosed in U.S. Pat. No. 4,275,149. The enzyme generally catalyses a chemical alteration of the chromogenic substrate that can be detected microscopically, e.g. under visible light. For example, the enzyme may catalyse a colour change in a substrate, or may alter the fluorescence or chemiluminescence of the substrate. Examples of enzymatic labels include luciferases (e.g. firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are well known.

Typically the method comprises a step of detecting stained regions within the image. Pixels in the image corresponding to staining associated with the biomarker may be identified by colour transformation methods, for instance as disclosed in U.S. Pat. Nos. 6,553,135 and 6,404,916. In such methods, stained objects of interest may be identified by recognising the distinctive colour associated with the stain. The method may comprise transforming pixels of the image to a different colour space, and applying a threshold value to suppress background staining. For instance, a ratio of two of the RGB signal values may be formed to provide a means for discriminating colour information. A particular stain may be discriminated from background by the presence of a minimum value for a particular signal ratio. For instance pixels corresponding to a predominantly red stain may be identified by a ratio of red divided by blue (R/B) which is greater than a minimum value.

Kong et al., Am J Clin Nutr, 2013 December; 98(6):1385-94 describes the use of the avidin-biotin-peroxidase method and two independent investigators counting the number of positively stained cells.

A detection using aptamers may comprise the following steps:
Aptamers that specifically recognize the biomarker may be synthesized using standard nucleic acid synthesis techniques or selected from a large random sequence pool, for example using the Systematic Evolution of Ligands by Exponential Enrichment (SELEX) technique;
Aptamers are mixed with the samples so that aptamer-protein complex are formed;
Non-specific complexes are separated;
Bound aptamers are removed from their target proteins;
Aptamers are collected and measured, for example using microarrays or mass spectrometry techniques.

Aptamers can be single strand DNA or RNA sequences that fold in a unique 3D structure having a combination of stems, loops, quadruplexes, pseudoknots, bulges, or hairpins. The molecular recognition of aptamers results from intermolecular interactions such as the stacking of aromatic rings, electrostatic and van der Waals interactions, or hydrogen bonding with a target compound. In addition, the specific interaction between an aptamer and its target is complemented through an induced fit mechanism, which requires the aptamer to adopt a unique folded structure to its target. Aptamers can be modified to be linked with labeling molecules such as dyes, or immobilized on the surface of beads or substrates for different applications.

In one embodiment, the biomarker level is compared with a reference value. In which case, the biomarker level in the sample and the reference value are determined using the same analytical method.

### Biomarkers

### Vitamin K-Dependent Protein C

Vitamin K-dependent protein C is the zymogenic form of protein C that circulates in blood plasma. Its structure is that of a two-chain polypeptide consisting of a light chain and a heavy chain connected by a disulfide bond. The activated form of vitamin K-dependent protein C plays an important role in regulating anticoagulation, inflammation, cell death, and maintaining the permeability of blood vessel walls. Activated protein C (APC) performs these operations primarily by proteolytically inactivating proteins Factor Va and Factor VIIIa. APC is classified as a serine protease.

Inactive vitamin K-dependent protein C is formed of 419 amino acids in multiple domains: one Gla domain (residues 43-88); a helical aromatic segment (89-96); two epidermal growth factor (EGF)-like domains (97-132 and 136-176); an activation peptide (200-211); and a trypsin-like serine protease domain (212-450). The light chain contains the Gla- and EGF-like domains and the aromatic segment. The heavy chain contains the protease domain and the activation peptide. It is in this form that 85-90% of protein C circulates in the plasma as a zymogen.

Vitamin K-dependent protein C is activated when it binds to thrombin, and protein C's activation is greatly promoted by the presence of thrombomodulin and endothelial protein C receptors (EPCRs). APC is formed when a dipeptide of Lys198 and Arg199 is removed; this causes the transformation into a heterodimer with N-linked carbohydrates on each chain. The protein has one light chain (21 kDa) and one heavy chain (41 kDa) connected by a disulfide bond between Cys183 and Cys319.

Methods for determining the level of vitamin K-dependent protein C in a sample are known in the art. Available methods for quantifying vitamin K-dependent protein C antigen levels include ELISA, immunodiffusion, and immunoelectrophoresis methods. For example, Kościelniak et al. describe the use of a commercially available ELISA to determine vitamin K-dependent protein C concentrations in a sample (Adv Clin Exp Med. 2015 September-October; 24(5):791-800).

In addition, functional assays may be used to determine the level of vitamin K-dependent protein C activity in a sample. Functional assays can either be clot-based or chromogenic (spectrophotometric) in design. Clot-based protein C assays use either a modified activated partial thromboplastin time (aPTT) or Russell viper venom (RVV) method on a plasma specimen treated with an activator of protein C. Clot-based assay results may be determined as clotting time (in seconds) and the time to clot formation is inversely related to protein C function, because protein C prolongs the clotting time by degrading factors V and VIII (e.g. see Moore et al.; Int J Lab Hematol. 2015 December; 37(6):844-52). Chromogenic assays detect the enzymatic activity of protein C by measuring cleavage of a specific chromogenic substrate by protein C, which results in colour generation that is directly proportional to the amount of protein C. Levels of vitamin K-dependent protein C in a sample may therefore be expressed as enzyme units/ml (e.g. see Kościelniak et al., as above).

In one embodiment, an increase in the level of vitamin K-dependent protein C in the sample relative to a reference value is indicative of a greater degree of weight loss in a subject.

An example human vitamin K-dependent protein C is the human vitamin K-dependent protein C having the UniProtKB accession number P04070. This exemplified sequence is 461 amino acids in length, of which amino acids 1 to 18 form a leader sequence.

### Coagulation Factor XIII A Chain

Coagulation factor XIII (fibrin stabilizing factor) is an enzyme (EC 2.3.2.13) of the blood coagulation system that crosslinks fibrin.

Coagulation factor XIII is a transglutaminase that circulates in the plasma as a heterotetramer of two catalytic A subunits (A polypeptide) and two carrier B subunits (B polypeptide). The genes for the A polypeptide and B polypeptide are encoded at separate loci in humans (A subunit (6p25-p24) and B subunit (1q31-q32.1)). The transglutaminase activity is provided by the A chain, whilst the B chain has no clear enzymatic activity, and may serve as a carrier for the A subunit.

When thrombin has converted fibrinogen to fibrin, the latter forms a proteinaceous network in which every E-unit is crosslinked to only one D-unit. In the presence of calcium as a cofactor, coagulation factor XIII is activated by thrombin into factor XIIIa. A cleavage by thrombin between residue Arg37 and Gly38 on the N-terminus of the coagulation factor XIII A subunit, leads to the release of the activation peptide. In the presence of calcium the carrier subunits dissociate from the catalytic subunits, leading to a conformational change of factor XIII and hence the exposure of catalytic cysteine residue. Upon activation by thrombin, factor XIIIa acts on fibrin to form γ-glutamyl-ϵ-lysyl amide cross links between fibrin molecules to form an insoluble clot.

As used herein, the term coagulation factor XIII A chain may refer to the coagulation factor XIII A polypeptide alone or in the context of the heterotetramer of two A subunits and two carrier B subunits. In one embodiment, the term coagulation factor XIII refers to the coagulation factor XIII A polypeptide.

Methods for determining the level of coagulation factor XIII A chain in a sample are known in the art. Coagulation factor XIII A chain levels may be determined by measuring levels of the coagulation factor XIII heterotetramer antigen or coagulation factor XIII A chain polypeptide antigen.

For example, Katona et al. describes an ELISA method for determining the concentration of the coagulation factor XIII heterotetramer antigen in a sample (Thromb Haemost. 2000 February 83(2):268-73). Ajzner et al. describes methods for determining the concentration of the coagulation factor XIII heterotetramer antigen; coagulation factor XIII A polypeptide antigen or coagulation factor XIII B polypeptide antigen in a sample (Blood: 2009; 113 (3)).

In addition, chromogenic functional assays may be used to determine the level coagulation factor XIII activity in a sample. For example, Karpati et al. describes an assay which involves monitoring the amount of ammonia (NH₃) released by using glutamate dehydrogenase and nicotinamide adenine dinucleotide phosphate during a transamidation reaction (cross-linking) by coagulation factor XIII (Clin Chem. 2000 December 46(12):1946-55). Levels of coagulation factor XIII in a sample may therefore be expressed as enzyme units/ml.

In one embodiment, an increase in the level of coagulation factor XIII A chain in the sample relative to a reference value is indicative of a greater degree of weight loss in a subject.

An example human coagulation factor XIII A polypeptide is the human coagulation factor XIII A polypeptide having the UniProtKB accession number P00488. This exemplified sequence is 732 amino acids in length, of which amino acid 1 is an initiator methionine.

An example human coagulation factor XIII B polypeptide is the human coagulation factor XIII B polypeptide having the UniProtKB accession number P05160. This exemplified sequence is 661 amino acids in length, of which amino acids 1 to 20 form a leader sequence.

### Pigment Epithelium-Derived Factor

Pigment epithelium-derived factor (PEDF) (also known as serpin F1 (SERPINF1)), is a multifunctional secreted protein that has anti-angiogenic, anti-tumorigenic, and neurotrophic functions. In humans, pigment epithelium-derived factor is encoded by the SERPINF1 gene.

Methods for determining the level of pigment epithelium-derived factor in a sample are known in the art. For example, Wang et al. describe the use of a commercially available ELISA assay from BioVendor Laboratory Medicine to determine the concentration of pigment epithelium-derived factor protein levels in a sample (Cardiovascular Diabetology; 2013; 12:56). Li et al. also describe the use of a commercially available ELISA to determine pigment epithelium-derived factor protein levels in serum samples (International Journal of COPD; 2015; 10; 587-594).

In one embodiment, an increase in the level of pigment epithelium-derived factor in the sample relative to a reference value is indicative of a greater degree of weight loss in a subject.

An example human pigment epithelium-derived factor is the human pigment epithelium-derived factor having the UniProtKB accession number P36955. This exemplified sequence is 418 amino acids in length, of which amino acids 1 to 19 form a leader sequence.

### Serum Amyloid P-Component

Serum amyloid P-component (SAP) is the identical serum form of amyloid P component (AP), a 25 kDa pentameric protein first identified as the pentagonal constituent of in vivo pathological amyloid deposits. Serum amyloid P-component is a member of the pentraxins family, characterised by calcium dependent ligand binding and distinctive flattened β-jellyroll structure similar to that of the legume lectins. Serum amyloid P-component may scavenge nuclear material released from damaged circulating cells and may also function as a calcium-dependent lectin.

Methods for determining the level of serum amyloid P-component are known in the art. For example, Tennant et al. describe a method for measuring serum amyloid P-component concentrations using an electroimmunoassay (Arthritis Rheum.; 2007; 56(6):2013-7). In addition, Bijl et al. describe the use of an ELISA to measure serum amyloid P-component concentrations (Ann Rheum Dis. 2004 July; 63(7):831-5).

In one embodiment, an increase in the level of serum amyloid P-component in the sample relative to a reference value is indicative of a greater degree of weight loss in a subject.

An example human serum amyloid P-component is the human serum amyloid P-component having the UniProtKB accession number P02743. This exemplified sequence is 223 amino acids in length, of which amino acids 1 to 19 form a leader sequence.

### Neuroligin-4, X-Linked

Neuroligin-4, X-linked is a member of the neuroligin family of neuronal cell surface proteins. Neuroligins may act as splice site-specific ligands for beta-neurexins. Neuroligin-4, X-linkedprotein interacts with discs, large (Drosophila) homolog 4 (DLG4).

Methods for determining the level of Neuroligin-4, X-linked in a sample are known in the art. For example, LifeSpan BioSciences, Inc. provide a commercial ELISA for determining concentrations of Neuroligin-4, X-linked in a sample (LifeSpan BioSciences, Inc., Product Code LS-F12160).

In one embodiment, an increase in the level of neuroligin-4, X-linked in the sample relative to a reference value is indicative of a greater degree of weight loss in a subject.

An example human Neuroligin-4, X-linked is the human Neuroligin-4, X-linked having the UniProtKB accession number Q8N0W4. This exemplified sequence is 816 amino acids in length, of which amino acids 1 to 41 form a leader sequence.

### CD226 Antigen

CD226 antigen is also known as ‘platelet and T cell activation antigen 1’ (PTA1) or DNAX Accessory Molecule-1 (DNAM-1).

CD226 antigen is a ˜65 kDa glycoprotein expressed on the surface of natural killer cells, platelets, monocytes and a subset of T cells. It is a member of the immunoglobulin superfamily containing 2 Ig-like domains of the V-set. CD226 antigen mediates cellular adhesion to other cells bearing its ligands, CD112 and CD15, and cross-linking CD226 antigen with antibodies causes cellular activation.

Methods for determining the level of CD226 antigen in a sample are known in the art. For example, LifeSpan BioSciences, Inc. provide a commercial ELISA for determining concentrations of CD226 antigen in a sample (LifeSpan BioSciences, Inc., Product Code LS-F7907).

In one embodiment, an increase in the level of CD226 antigen in the sample relative to a reference value is indicative of a greater degree of weight loss in a subject.

An example human CD226 antigen is the human CD226 antigen having the UniProtKB accession number Q15762. This exemplified sequence is 336 amino acids in length, of which amino acids 1 to 18 form a leader sequence.

### Metalloproteinase Inhibitor 3

Metalloproteinase inhibitor 3 belongs to the family of tissue inhibitor of metalloproteinases. The proteins encoded by this gene family are inhibitors of the matrix metalloproteinases, a group of peptidases involved in degradation of the extracellular matrix (ECM). Expression of metalloproteinase inhibitor 3 is localized to the ECM and is induced in response to mitogenic stimulation.

Methods for determining the level of metalloproteinase inhibitor 3 in a sample are known in the art. For example, Cheng et al. describe the use of a commercially available ELISA to measure plasma concentrations of metalloproteinase inhibitor 3 (Int J Biol Sci. 2013 Jun. 12; 9(6):557-63). In addition, Valbuena-Diez et al. describe the use of an alternative commercially available ELISA for determining the concentration of metalloproteinase inhibitor 3 in a sample (Circulation. 2012 Nov. 27; 126(22):2612-24).

In one embodiment an increase in the level of metalloproteinase inhibitor 3 in the sample relative to a reference value is indicative of a greater degree of weight loss in a subject.

An example human metalloproteinase inhibitor 3 is the human metalloproteinase inhibitor 3 having the UniProtKB accession number P35625. This exemplified sequence is 211 amino acids in length, of which amino acids 1 to 23 form a leader sequence.

### Eukaryotic Translation Initiation Factor 4E-Binding Protein 2

Eukaryotic translation initiation factor 4E-binding protein 2 is a member of a family of translation repressor proteins. The protein directly interacts with eukaryotic translation initiation factor 4E (eIF4E), which is a limiting component of the multisubunit complex that recruits 40S ribosomal subunits to the 5′ end of mRNAs. Interaction of this protein with eIF4E inhibits complex assembly and represses translation. This protein is phosphorylated in response to various signals including UV irradiation and insulin signaling, resulting in its dissociation from eIF4E and activation of cap-dependent mRNA translation.

Methods for determining the level of eukaryotic translation initiation factor 4E-binding protein 2 in a sample are known in the art. For example, MyBioSource.com provide a commercial ELISA for determining concentrations of eukaryotic translation initiation factor 4E-binding protein 2 in a sample (MyBioSource.com; Product Code MBS9325222).

In one embodiment, an increase in the level of eukaryotic translation initiation factor 4E-binding protein 2 in the sample relative to a reference value is indicative of a greater degree of weight loss in a subject.

An example human eukaryotic translation initiation factor 4E-binding protein 2 is the human eukaryotic translation initiation factor 4E-binding protein 2 having the UniProtKB accession number Q13542. This exemplified sequence is 120 amino acids in length.

### Leukocyte Immunoglobulin-Like Receptor Subfamily B Member 2

Leukocyte immunoglobulin-like receptor (LIR) subfamily B member 2 (also known as ILT-4) belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. The receptor can also function in antigen capture and presentation.

Methods for determining the level of leukocyte immunoglobulin-like receptor subfamily B member 2 in a sample are known in the art. For example, Baffari et al. describe a method for determining the levels of leukocyte immunoglobulin-like receptor subfamily B member 2 in a sample (Hum Immunol. 2013 October; 74(10):1244-50).

In one embodiment, a decrease in the level of leukocyte immunoglobulin-like receptor subfamily B member 2 in the sample relative to a reference value is indicative of a greater degree of weight loss in a subject.

An example human leukocyte immunoglobulin-like receptor subfamily B member 2 is the human leukocyte immunoglobulin-like receptor subfamily B member 2 having the UniProtKB accession number Q8N423. This exemplified sequence is 598 amino acids in length, of which amino acids 1 to 21 form a leader sequence.

### X-Ray Repair Cross-Complementing Protein 6

X-ray repair cross-complementing protein 6 (Ku70) forms a complex with X-ray repair cross-complementing protein 5 (Ku80) to make up the Ku heterodimer, which binds to DNA double-strand break ends and is required for the non-homologous end joining (NHEJ) pathway of DNA repair. It is also required for V(D)J recombination, which utilizes the NHEJ pathway to promote antigen diversity in the mammalian immune system. In addition to its role in NHEJ, Ku is also required for telomere length maintenance and subtelomeric gene silencing.

Methods for determining the level of X-ray repair cross-complementing protein 6 in a sample are known in the art. For example, LifeSpan BioSciences, Inc. provide a commercial ELISA for determining the concentration of X-ray repair cross-complementing protein 6 in a sample (LifeSpan BioSciences, Inc; Sandwich ELISA; Product Code LS-F4618).

In one embodiment, a decrease in the level of X-ray repair cross-complementing protein 6 in the sample relative to a reference value is indicative of a greater degree of weight loss in a subject.

An example human X-ray repair cross-complementing protein 6 is the human X-ray repair cross-complementing protein 6 having the UniProtKB accession number P12956. This exemplified sequence is 609 amino acids in length, of which amino acid 1 is an initiator methionine.

### Caspase-2

Sequential activation of caspases plays a central role in the execution-phase of cell apoptosis. Caspases exist as inactive proenzymes that undergo proteolytic processing at conserved aspartic residues to produce two subunits, large and small, that dimerize to form the active enzyme.

Caspase 2 is part of the Ich-1 subfamily and is one of the most conserved caspases. Caspase 2 has a similar amino acid sequence to initiator caspases, including caspase 1, caspase 4, caspase 5, and caspase 9. It is produced as a zymogen, which contains a long pro-domain that is similar to that of caspase 9 and contains a protein interaction domain known as a CARD domain. Pro-caspase-2 contains two subunits, p19 and p12. Caspase 2 has been shown to associate with several proteins involved in apoptosis via its CARD domain, including RIP-associated Ich-1/Ced-3-homologue protein with a death domain (RAIDD), apoptosis repressor with caspase recruitment domain (ARC), and death effector filament-forming Ced-4-like apoptosis protein (DEFCAP).

Methods for determining the level of caspase-2 in a sample are known in the art. For example, levels of caspase-2 in a sample may be determined by functional chromogenic (spectrophotometric) assays. For example, Promega provide a commercial caspase-2 assay based on a Z-VDVAD-aminoluciferin substrate. Caspase-2 mediated cleavage of the substrate results in the generation of a glow-type luminescent signal produced by luciferase, wherein the luminescence is proportional to the amount of caspase-2 activity present in the sample (Promega, Product Code G0940 and G0941, see e.g. U.S. Pat. No. 7,148,030). Levels of caspase-2 in a sample may therefore be expressed as enzyme units/ml.

In one embodiment, a decrease in the level of caspase-2 in the sample relative to a reference value is indicative of a greater degree of weight loss in a subject.

An example human caspase 2 is the human caspase 2 having the UniProtKB accession number P42575-1. This exemplified sequence is 452 amino acids in length, of which amino acid 1 is an initiator methionine. A second example human caspase 2 is the human caspase 2 having the UniProtKB accession number P42575-2. This exemplified sequence is 292 amino acids in length.

### Interleukin-34

Interleukin-34 (IL-34) binds to Colony stimulating factor 1 receptor (CSF1R) and increases growth or survival of monocytes and macrophages. It promotes the release of proinflammatory chemokines, and thereby plays an important role in innate immunity and in inflammatory processes. Signaling via CSF1R and its downstream effectors stimulates phosphorylation of MAPK1/ERK2 AND MAPK3/ERK1.

Methods for determining the level of Interleukin-34 in a sample are known in the art. For example, BioLegend® provide a commercial ELISA for determining the concentration of interleukin-34 in a sample (BioLegend®, Product Code #439607). In addition, Tian et al. describe the use of a further commercial ELISA to determine the concentration of interleukin-34 in a sample (J Interferon Cytokine Res. 2013 July; 33(7):398-401).

In one embodiment, a decrease in the level of Interleukin-34 in the sample relative to a reference value is indicative of a greater degree of weight loss in a subject.

An example human interleukin-34 is the human interleukin-34 having the UniProtKB accession number Q6ZMJ4. This exemplified sequence is 242 amino acids in length, of which amino acids 1 to 20 form a leader sequence.

### Interleukin-17 Receptor C

Interleukin-17 receptor C (IL17RC) is a single-pass transmembrane protein that shares limited similarity with the interleukin-17 receptor A. Unlike Interleukin-17 receptor A (IL-17RA), which is predominantly expressed in hemopoietic cells, and binds with high affinity to only IL-17A, interleukin-17 receptor C is expressed in nonhemopoietic tissues, and binds both IL-17A and IL-17F with similar affinities. The proinflammatory cytokines, IL-17A and IL-17F, have been implicated in the progression of inflammatory and autoimmune diseases.

Methods for determining the level of human interleukin-17 receptor C in a sample are known in the art. For example, Wu et al. describe a method for determining levels of human interleukin-17 receptor C using immunohistochemistry and flow cytometry methods (J Transl Med. 2014 May 31; 12:152).

In one embodiment, a decrease in the level of interleukin-17 receptor C in the sample relative to a reference value is indicative of a greater degree of weight loss in a subject.

An example human interleukin-17 receptor C is the human interleukin-17 receptor C having the UniProtKB accession number Q8NAC3. This exemplified sequence is 791 amino acids in length, of which amino acids 1 to 20 form a leader sequence.

### Protein Z-Dependent Protease Inhibitor

Protein Z-dependent protease inhibitor (also known as Serpin 10) is encoded by the SERPINA10 gene. It inhibits the activity of the coagulation protease factor Xa in the presence of “protein Z, vitamin K-dependent plasma glycoprotein”, calcium and phospholipids and also inhibits factor XIa in the absence of cofactors.

Methods for determining the level of protein Z-dependent protease inhibitor in a sample are known in the art. For example, Kim et al. describe the use of an ELISA to determine protein Z-dependent protease inhibitor levels in plasma samples (Journal of Gastroenterology and Hepatology; 2015; 30; 4:784-793). Al-Shanqeeti et al. also describe the use of an ELISA to determine protein Z-dependent protease inhibitor levels in plasma samples (Thrombosis and Haemostasis; 2005; 93:3; 399-623).

In one embodiment, an increase in the level of protein Z-dependent protease inhibitor in the sample relative to a reference value is indicative of a greater degree of weight loss in a subject.

An example human protein Z-dependent protease inhibitor is the human Protein Z-dependent protease inhibitor having the UniProtKB accession number Q9UK55. This exemplified sequence is 444 amino acids in length, of which amino acids 1 to 21 form a leader sequence.

### Serum Paraoxonase/Arylesterase 1

The paraoxonase (PON) enzyme family comprises three members, PON1, PON2, and PON3, whose genes are located adjacent to each other on chromosome 7q21-2. PONs are able to retard low-density lipoprotein (LDL) oxidation and cellular oxidative stress.

In humans, PON1 and PON3 genes are produced in many cell types, and their protein products are found in the circulation bound to high density lipoprotein (HDL) (Sierksma et al.; Alcohol. Clin. Exp. Res. 26: 1430-1435).

Serum paraoxonase/arylesterase 1 (PON1) is also known in the art as aromatic esterase 1 or serum aryldialkylphosphatase 1. It has both paraoxonase and arylesterase activities. PON1 is a HDL-associated enzyme which has been reported to prevent the accumulation of lipoperoxides and LDL (van Himbergen et al; Neth J Med. 2006 February; 64(2):34-8).

PON1 protein levels may be determined using commercially available immunoassays. For example, Loued et al. describe the use of an immunoassay from Uscn Life Science, Inc. to determine the concentration of PON1 protein levels in a sample (Loued et al.; British Journal of Nutrition (2013); 110; 1272-128).

PON levels may be determined as enzyme activity levels. For example, methods for determining activity levels of PON1 are known in the art.

PON1 paraoxonase activity may be determined by the rate of enzymatic hydrolysis of paraoxon (O,O-diethyl-O-p-nitrophenylphosphate) to p-nitrophenol, for example as described by Dursun et al. (Human Reproduction Vol. 21, No. 1 pp. 104-108, 2006). The level of p-nitrophenol may be measured using a spectrophotometer by determining the increase in absorbance at 412 nm.

PON1 arylesterase activity may be determined by the rate of enzymatic hydrolysis of phenylacetate by measuring absorbance on a spectrometer at 270 nm, for example as described by Teiber et al. (Clinical Chemistry August 2013 vol. 59 no. 8 1251-1259).

In one embodiment, an increase in the level of serum paraoxonase/arylesterase 1 in the sample relative to a reference value is indicative of a greater degree of weight loss in a subject.

An example human PON1 protein is the human PON1 protein having the UniProtKB accession number P27169. This exemplified sequence is 355 amino acids in length of which amino acid 1 is an initiator methionine.

### Plasminogen

Plasminogen is the zymogen form of plasmin which is released from the liver into the factor IX systemic circulation. In circulation, plasminogen adopts a closed, activation resistant conformation. Upon binding to clots, or to the cell surface, plasminogen adopts an open form that can be converted into active plasmin by a variety of enzymes, including tissue plasminogen activator (tPA), urokinase plasminogen activator (uPA), kallikrein, and factor XII (Hageman factor). Fibrin is a cofactor for plasminogen activation by tissue plasminogen activator. Urokinase plasminogen activator receptor (uPAR) is a cofactor for plasminogen activation by urokinase plasminogen activator. The conversion of plasminogen to plasmin involves the cleavage of the peptide bond between Arg-561 and Val-562.

Activated plasmin degrades many blood plasma proteins, including fibrin clots via a process termed fibrinolysis. Apart from fibrinolysis, plasmin proteolyses proteins in various other systems: It activates collagenases, some mediators of the complement system and weakens the wall of the Graafian follicle.

Methods for determining the level of plasminogen in a sample are known in the art. For example, Banville et al., describe the use of a commercially available ELISA for determining plasminogen concentrations in a sample (PLoS One. 2014 Aug. 18; 9(8):e105365). In addition, levels of plasminogen in a sample may be determined by functional chromogenic (spectrophotometric) assays. For example, plasmin enzyme activity may be determined using a plasmin-specific chromogenic substrate (e.g. Tait et al; Thromb Haemost. 1992 Nov. 10; 68(5):506-10). Levels of plasminogen in a sample may therefore be expressed as enzyme units/ml.

In one embodiment, a decrease in the level of plasminogen in the sample relative to a reference value is indicative of a greater degree of weight loss in a subject.

An example human plasminogen protein is the human plasminogen protein having the UniProtKB accession number P00747. This exemplified sequence is 810 amino acids in length of which amino acids 1 to 19 form a leader sequence.

### Complement Factor D

Complement factor D is involved in the alternative complement pathway of the complement system where it cleaves factor B. It is a member of the chymotrypsin family of serine proteases and has a structure comprising two antiparallel β-barrel domains with each barrel containing six β-strands. The major difference in backbone structure between Factor D and the other serine proteases of the chymotrpsin family is in the surface loops connecting the secondary structural elements. Factor D displays different conformations of major catalytic and substrate-binding residues typically found in the chrotrypsin family. These features suggest the catalytic activity of factor D is prohibited unless conformational changes are induced by a realignment.

Methods for determining the level of complement factor D in a sample are known in the art. For example, Glasgow et al., describe the use of a commercially available ELISA to determine the concentration of complement factor D in a sample (Chest. 2009 May; 135(5): 1293-1300).

In one embodiment, an increase in the level of complement factor D in the sample relative to a reference value is indicative of a greater degree of weight loss in a subject.

An example human complement factor D protein is the human complement factor D protein having the UniProtKB accession number P00746. This exemplified sequence is 253 amino acids in length of which amino acids 1 to 20 form a leader sequence.

### Leptin

Leptin is a hormone made by adipose cells that helps to regulate energy balance by inhibiting hunger. Leptin is opposed by the actions of the hormone ghrelin and both hormones act on receptors in the arcuate nucleus of the hypothalamus to regulate appetite to achieve energy homeostasis. In obesity, a decreased sensitivity to leptin occurs, resulting in an inability to detect satiety despite high energy stores.

Although regulation of fat stores is deemed to be the primary function of leptin, it also plays a role in other physiological processes, as evidenced by its multiple sites of synthesis other than fat cells, and the multiple cell types beside hypothalamic cells that have leptin receptors. Methods for determining the level of leptin in a sample are known in the art. For example, Behnes et al. describe the use of a commercially available ELISA for determining the concentration of leptin in a sample (BMC Infect Dis. 2012 Sep. 14; 12:217).

In one embodiment a decrease in the level of leptin in the sample relative to a reference value is indicative of a greater degree of weight loss in a subject.

An example human leptin protein is the human leptin protein having the UniProtKB accession number P41159. This exemplified sequence is 167 amino acids in length of which amino acids 1 to 21 form a leader sequence.

### Carbonic Anhydrase 6

Carbonic anhydrase 6 (also called Gustin) is one of several isozymes of carbonic anhydrase. This protein is abundantly found in salivary glands and saliva and may play a role in the reversible hydratation of carbon dioxide.

Methods for determining the level of carbonic anhydrase 6 in a sample are known in the art. For example, Sim et al. describe the use of a commercially available ELISA to determine the concentration of carbonic anhydrase 6 in a sample (Br J Cancer. 2012 Sep. 25; 107(7):1131-7).

In one embodiment, an increase in the level of carbonic anhydrase 6 in the sample relative to a reference value is indicative of a greater degree of weight loss in a subject.

An example human carbonic anhydrase 6 protein is the human carbonic anhydrase 6 protein having the UniProtKB accession number P23280. This exemplified sequence is 308 amino acids in length of which amino acids 1 to 17 form a leader sequence.

### Macrophage Metalloelastase

Macrophage metalloelastase (also known as Matrix metalloproteinase-12 (MMP-12)) is a member of the matrix metalloproteinase (MMP) family of proteins. These proteins are involved in the breakdown of extracellular matrix in normal physiological processes, such as embryonic development, reproduction, and tissue remodeling, as well as in disease processes, such as arthritis and metastasis. Macrophage metalloelastase is secreted as inactive proprotein which is cleaved by extracellular proteinases in order to activate the enzyme. The active enzyme is constituted by two domains, the catalytic domain responsible for its enzymatic activity and the hemopexin-like domain.

Macrophage metalloelastase catalyses the hydrolysis of soluble and insoluble elastin. It also specifically cleaves between Ala-14/Leu-15 and Tyr-16/Leu-17 in the B chain of insulin.

Methods for determining the level of macrophage metalloelastase in a sample are known in the art. For example, Manetti et al. describe the use of a commercially available ELISA to determine the concentration macrophage metalloelastase in a sample (Ann Rheum Dis 2012; 71:A48). In addition, levels of macrophage metalloelastase in a sample may be determined by functional chromogenic (spectrophotometric) assays. For example, macrophage metalloelastase enzyme activity may be determined using a specific chromogenic substrate (e.g. Yokose et al; Arthritis & Rheumatism; 62(8); 2488-2498; 2010). Levels of macrophage metalloelastase in a sample may therefore be expressed as enzyme units/ml.

In one embodiment, a decrease in the level of macrophage metalloelastase in the sample relative to a reference value is indicative of a greater degree of weight loss in a subject.

An example human macrophage metalloelastase protein is the human macrophage metalloelastase protein having the UniProtKB accession number P39900. This exemplified sequence is 470 amino acids in length of which amino acids 1 to 16 form a leader sequence.

### Angiopoietin-1

Angiopoietins are proteins with important roles in vascular development and angiogenesis. All angiopoietins bind with similar affinity to an endothelial cell-specific tyrosine-protein kinase receptor. Angiopoietin-1 is a secreted glycoprotein that activates the receptor by inducing its tyrosine phosphorylation. It plays a critical role in mediating reciprocal interactions between the endothelium and surrounding matrix and mesenchyme. Angiopoietin-1 also contributes to blood vessel maturation and stability, and may be involved in early development of the heart.

Methods for determining the level of angiopoietin-1 in a sample are known in the art. For example, Schreitmüller et al. describe the use of a commercially available ELISA to determine the concentration of angiopoietin-1 in a sample (International Journal of Alzheimer's Disease; 2012, Article ID 324016).

In one embodiment, an increase in the level of angiopoietin-1 in the sample relative to a reference value is indicative of a greater degree of weight loss in a subject.

An example human angiopoietin-1 protein is the human angiopoietin-1 protein having the UniProtKB accession number Q15389. This exemplified sequence is 498 amino acids in length of which amino acids 1 to 15 form a leader sequence.

### Combinations of Biomarkers

Whilst individual biomarkers may have predictive value in the methods of the present invention, the quality and/or the predictive power of the methods may be improved by combining values from multiple biomarkers.

Thus the present method may involve determining the level of at least two biomarkers from those described herein. For instance, the method may comprise determining the level of two or more biomarkers selected from vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

The term “one or more biomarkers” as used herein may include one, two, three, four, five, six, seven, eight, nine, ten, eleven, twelve, thirteen, fourteen, fifteen, sixteen, seventeen, eighteen, nineteen or twenty biomarkers as described herein.

### Comparison to a Reference or Control

The present method may further comprise a step of comparing the level of the individual biomarkers in the test sample to one or more reference or control values. The reference value may be associated with a pre-defined ability of a subject to lose weight following dietary intervention. In some embodiments, the reference value is a value obtained previously for a subject or group of subjects following a certain dietary intervention. The reference value may be based on an average level, e.g. a mean or median level, from a group of subjects following the dietary intervention.

Combining the Biomarker Levels with Anthropometric Measures and/or Lifestyle Characteristics

In one embodiment, the present method further comprises combining the level of the one or more biomarkers with one or more anthropometric measures and/or lifestyle characteristics of the subject. By combining this information, an improved predictive model is provided for the degree of weight loss attainable by a subject.

As is known in the art, an anthropometric measure is a measurement of a subject. In one embodiment, the anthropometric measure is selected from the group consisting of gender, age (in years), weight (in kilograms), height (in centimetres), and body mass index (in kg/m²). Other anthropometric measures will also be known to the skilled person in the art.

By the term “lifestyle characteristic” is meant any lifestyle choice made by a subject, this includes all dietary intake data, activity measures or data from questionnaires of lifestyle, motivation or preferences. In one embodiment, the lifestyle characteristic is whether the subject is a smoker or a non-smoker. This is also referred to herein as the smoking status of the subject.

In a preferred embodiment, levels of one or more biomarkers as defined herein are determined for a sample from the subject and these levels are combined with the gender, age, smoking status and body mass index of the subject in order to predict the weight loss attainable by the subject. Preferably the degree of weight loss is represented by the body mass index that a subject is predicted to attain by applying the dietary intervention.

In one embodiment, the predicted body mass index (BMI2) is generally represented by formula (1):

BMI2=c1*BMI1+c2*age−c3*vitamin K-dependent protein C−c4*coagulation factor XIII A chain−c5*pigment epithelium-derived factor−c6*serum amyloid P-component−c7*neuroligin-4,X-linked−c8*CD226 antigen−c9*metalloproteinase inhibitor 3−c10*eukaryotic translation initiation factor 4E-binding protein 2+c11*leukocyte immunoglobulin-like receptor subfamily B member 2+c12*X-ray repair cross-complementing protein 6+c13*caspase-2+c14*interleukin-34+c15*interleukin-17 receptor C−c16*protein Z-dependent protease inhibitor−c17*serum paraoxonase/arylesterase 1+c18*plasminogen−c19*complement factor D+c20*leptin−c21*carbonic anhydrase 6+c22*macrophage metalloelastase−c23*angiopoietin-1;

wherein BMI1 is the subject's body mass index before the dietary intervention and BMI2 is the subject's predicted body mass index after the dietary intervention; and wherein c1 to c23 are positive integers.

The values of c1 to c23 typically depend on 1) the measurement units of all the variables in the model; and 2) provenance (ethnic background) of the considered subject. Each of the coefficients c1 to c23 can be readily determined for particular subject cohorts. As would be understood by the skilled person, a dietary intervention, for example a low calorie diet, may be applied to a subject cohort of interest, the levels of the biomarkers as defined herein may be determined and routine statistical methods may then be used in order to arrive at the values of c1 to c7. Such routine statistical methods may include multiple linear regression with calibration by bootstrap. It is possible to obtain the same estimates with generalized linear or additive models or any other regression-related model with various estimation algorithms, for example, elastic net, lasso, Bayesian approach etc.

In one embodiment, the subject is European.

### Subject Stratification

The degree of weight loss predicted by the method of the present invention may also be compared to one or more pre-determined thresholds. Using such thresholds, subjects may be stratified into categories which are indicative of the degree of predicted weight loss, e.g. low, medium, high and/or very high predicted degree of weight loss. The extent of the divergence from the thresholds is useful to determine which subjects would benefit most from certain interventions. In this way, dietary intervention and modification of lifestyle can be optimised, and realistic expectations of the weight loss to be achieved by the subject can be set.

In one embodiment, the categories include weight loss resistant subjects and weight loss sensitive subjects.

By the term “weight loss resistant” is meant a predicted degree of weight loss which is less than a predetermined value. Preferably “weight loss resistant” is defined as a subject having a weight loss percentage inferior to a predetermined value e.g. a subject predicted to lose less weight than the 10^{th}, 15^{th}, 20^{th} or 30^{th} percentile of the expected weight loss for the subject.

Preferably the degree of weight loss is represented by the number of BMI units lost, where BMI loss=((BMI1−BMI2)*100)/BMI1, wherein BMI1 is the body mass index of the subject before the dietary intervention and BMI2 is the predicted body mass index of the subject after the dietary intervention.

By the term “weight loss sensitive” is meant a predicted degree of weight loss of more than a predetermined value. Preferably “weight loss sensitive” is defined as a subject having a weight loss percentage superior to a predetermined threshold value. For example a subject predicted to lose more weight than the 85^{th}, 80^{th} or 75^{th} percentile of the expected weight loss.

The “expected weight loss” can be obtained from data of a population of subjects that have undergone the same dietary intervention as the one being tested.

In another embodiment, subjects may be stratified into categories “weight loss sensitive” or “weight loss resistant” which are indicative of the risk reduction of the subject for obesity or obesity-related disorders, e.g. low, medium, high and/or very high risk reduction. Low, medium and high risk reduction groups may be defined in terms of absolute weight loss, where the absolute weight loss relates to clinical criteria for obesity or a particular obesity-related disorder.

For example, if the aim is to reduce the risk for type 2 diabetes in an obese individual, “very high risk reduction” may be defined as those predicted to lose at least 10% body weight after the dietary intervention. This is in accordance with the criteria set out in Part II of the World Health Organ Tech Rep Ser. 2000; 894:i-xii, 1-253). Moreover every 1% reduction in body weight of an obese person leads to a fall in systolic and diastolic blood pressure, and fall in low-density lipoprotein cholesterol, hence reduces the risk of cardio-vascular disease and dyslipidaemia respectively.

### Method for Selecting a Modification of Lifestyle of a Subject

In a further aspect, the present invention provides a method for modifying the lifestyle of a subject. The modification in lifestyle in the subject may be any change as described herein, e.g. a change in diet, more exercise, a different working and/or living environment etc.

Preferably the modification is a dietary intervention as described herein. More preferably the dietary intervention includes the administration of at least one diet product. The diet product preferably has not previously been consumed or was consumed in different amounts by the subject. The diet product may be as described herein. Modifying a lifestyle of the subject also includes indicating a need for the subject to change his/her lifestyle, e.g. prescribing more exercise or stopping smoking.

For example, if a subject is not predicted to lose weight on a low calorie diet, a modification may include more exercise in the subject's lifestyle.

### Use of Diet Products

In one aspect, the present invention provides a diet product for use as part of a low calorie diet for weight loss. The diet product being administered to a subject that is predicted to attain a degree of weight loss by the methods described herein.

In another aspect, the present invention provides a diet product for use in treating obesity or an obesity-related disorder, wherein the diet product is administered to a subject that is predicted to attain a degree of weight loss by the methods described herein.

The obesity-related disorder may be selected from the group consisting of diabetes (e.g. type 2 diabetes), stroke, high cholesterol, cardiovascular disease, insulin resistance, coronary heart disease, metabolic syndrome, hypertension and fatty liver. In a further aspect, the present invention provides the use of a diet product in a low calorie diet for weight loss where the diet product is administered to a subject that is predicted to attain a degree of weight loss by the methods described herein.

### Kits

In a further aspect, the present invention provides a kit for predicting the degree of weight loss attainable by applying one or more dietary interventions to the subject.

In one embodiment the kit comprises: (a) an antibody specific for vitamin K-dependent protein C; and (b) one ore more antibodies, each of which is specific one of coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase or angiopoietin-1.

In one embodiment the kit comprise at least two antibodies; wherein each antibody is specific for one of vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; complement factor D; leptin; carbonic anhydrase 6; macrophage metalloelastase or angiopoietin-1.

In one embodiment the kit comprise at least two antibodies; wherein each antibody is specific for one of vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; complement factor D; leptin; carbonic anhydrase 6; macrophage metalloelastase or angiopoietin-1.

The term antibody includes antibody fragments. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. Furthermore, the antibodies and fragments thereof may be humanised antibodies. The skilled person will be aware of methods in the art to produce the antibodies required for the present kit.

### Computer Program Product

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to predict the degree of weight loss based on the levels of biomarkers as described herein.

In another aspect, the present invention provides a computer program product comprising computer implementable instructions for causing a device to predict the degree of weight loss given the levels of one or more biomarkers from the user, wherein the biomarkers are selected from the one or more biomarkers as defined herein. Preferably the biomarker levels are fasting levels. The computer program product may also be given anthropometric measures and/or lifestyle characteristics from the user. As described herein, anthropometric measures include age, weight, height, gender and body mass index and lifestyle characteristics include smoking status.

In one embodiment, the user inputs into the device levels of one or more of the biomarkers as defined herein, optionally along with age, body mass index, gender and smoking status. The device then processes this information and provides a prediction on the degree of weight loss attainable by the user from a dietary intervention.

The device may generally be a server on a network. However, any device may be used as long as it can process biomarker data and/or anthropometric and lifestyle data using a processor, a central processing unit (CPU) or the like. The device may, for example, be a smartphone, a tablet terminal or a personal computer and output information indicating the degree of weight loss attainable by the user.

Those skilled in the art will understand that they can freely combine all features of the present invention described herein, without departing from the scope of the invention as disclosed.

Various preferred features and embodiments of the present invention will now be described by way of non-limiting examples.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O′D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and E. M. Shevach and W. Strober, 1992 and periodic supplements, Current Protocols in Immunology, John Wiley & Sons, New York, N.Y. Each of these general texts is herein incorporated by reference.

### EXAMPLES

### Example 1—Predicting Weight Loss after Low Calorie Diet (LCD) Using Blood Plasma Biomarkers

The Diogenes data set was used to identify proteins predicting the success of a subject to lose weight at the baseline of a Low Calorie Diet (LCD) with meal replacement (800 kCal per day). This study is a pan-European, randomised and controlled dietary intervention study investigating the effects of dietary protein and glycaemic index on weight loss and weight maintenance in obese and overweight families in roughly 500 subjects over eight European centres (Larsen et al., Obesity reviews (2009), 11, 76-91).

Fasting plasma was taken from all the participants shortly before their adherence to an eight week low caloric dietary intervention. The Diogenes data set includes 1240 proteins identified using either SomaLogic or LC-MS technology. Each data set was analysed separately to identify proteins predictive of weight loss success and obtain a predictive model for subject stratification at baseline.

Elastic net Zou and Hastie (2005; Journal of the Royal Statistical Society: Series B (Statistical Methodology) 67 (2): 301-20) with appropriately adapted Lasso Bootstrap (Chatterjee and Lahiri; 2011; Journal of the American Statistical Association 106 (494): 608-25) was used for statistical selection of most relevant variables within the publicly available R software (R Core Team; 2015; 2015. R: A Language and Environment for Statistical Computing. Vienna, Austria: R Foundation for Statistical Computing).

Anthropometric measures were also taken prior to the dietary intervention, including age, weight and height (from which the bmi—body mass index—is derived as weight/height²) and gender.

All the variables measured prior to the dietary intervention were evaluated for being separately and jointly predictors of bmi2 given bmi1. The inventors evaluated multiple statistical models using freely available tools (R software) and retained following predictive model:

bmi2=c1*bmi1+c2*age−c3*vitamin K dependent protein C−c4*coagulation factor XIII A chain−c5*pigment epithelium derived factor−c6*serum amyloid P component−c7*neuroligin4,X linked−c8*CD226 antigen−c9*metalloproteinase inhibitor 3−c10*eukaryotic translation initiation factor 4E binding protein 2+c11*leukocyte immunoglobulin like receptor subfamily B member 2+c12*Xray repair crosscomplementing protein 6+c13*caspase2+c14*interleukin34+c15*interleukin17 receptor C−c16*protein Z dependent protease inhibitor−c17*serum paraoxonase arylesterase 1+c18*plasminogen−c19*complement factor D+c20*leptin−c21*carbonic anhydrase 6+c22*macrophage metalloelastase−c23*angiopoietin1;

where coefficients c1 to c23 are positive and their values depend on 1) the measurement units of all the variables in the model; and 2) provenance (ethnic background) of the considered subject.

Using the estimation method proposed by MacLehose et al. (2007; Epidemiology (Cambridge, Mass.) 18 (2): 199-207), the significance of all the coefficients of the predictive model for the expected bmi2 (bmi after the end of low calorie diet) was demonstrated (Table 1).

TABLE 1
Coefficients when predicting average expected bmi2 with
regression as in (1) and Bayesian posterior probabilities
of corresponding coefficients to be greater than 0. Computations
use the model Bayesian regression model estimator proposed
by MacLehose et al. (P2 model).

| | Bayesian posterior probability for the |
|---|---|
| Coefficient for protein | coefficient to be greater than 0 |
| Vitamin K-dependent protein C | 0.62 |
| Coagulation factor XIII A chain | 0.70 |
| Pigment epithelium-derived factor | 0.50 |
| Serum amyloid P-component | 0.79 |
| Neuroligin-4, X-linked | 0.99 |
| CD226 antigen | 0.99 |
| Metalloproteinase inhibitor 3 | 0.98 |
| Eukaryotic translation initiation | 0.60 |
| factor 4E-binding protein 2 | |
| Leukocyte immunoglobulin-like | 0.57 |
| receptor subfamily B member 2 | |
| X-ray repair cross-complementing | 0.99 |
| protein 6 | |
| Caspase-2 | 0.99 |
| Interleukin-34 | 0.92 |
| Interleukin-17 receptor C | 0.82 |
| Protein Z-dependent protease | 0.77 |
| inhibitor | |
| Serum paraoxonase/arylesterase 1 | 0.65 |
| Plasminogen | 0.94 |
| Complement factor D | 0.89 |
| Leptin | 0.95 |
| Carbonic anhydrase 6 | 0.99 |
| Macrophage metalloelastase | 0.94 |
| Angiopoietin-1 | 0.69 |

### Example 2: Stratification of Subjects According to Predicted Weight Loss and Success Thresholds

The term “Obese Diet Resistant” (ODR) proposed by Ghosh et al. (2011; Obesity (Silver Spring) 19 (2): 457-63) is to be interpreted as being “weight loss resistant” in that a subject is predicted to have a weight loss percentage inferior to a pre-determined threshold value. As an example “weight loss resistance” may be defined as predicted to lose less bmi units than the 30th or 15th percentile of the expected bmi loss (where bmi loss=(bmi1−bmi2)*100%/bmi1).

The term “Obese Diet Sensitive” (ODS) is to be interpreted as being “weight loss sensitive” in that a subject is predicted to have a weight loss percentage superior to a pre-determined threshold value. As an example “weight loss sensitivity” may be defined as predicted to lose more bmi units than the 70th or 85th percentile of the expected bmi loss.

The expected average median or other percentiles of the bmi loss can be obtained by a skilled person on a sample of subjects (from the population of interest) that has undergone a dietary intervention similar to the one to be used.

Receiver Operating Characteristic (ROC) curve is a “best-developed statistical tool for describing the performance of” diagnostic tests measured on continuous scale (see Pepe, M. S. (2003). The Statistical Evaluation of Medical Tests for Classification and Prediction, Oxford University Press, New York, page 66). ROC use is based on the dichotomization of the test outcome. In our case we define the group of “weight loss resistant” subjects and predict the probability of subject to be in this group prior to the dietary intervention. We consider two definitions of “weight loss resistant” corresponding to losing less than 9% of initial weight or “weight loss sensitive” as losing more than 13% of initial weight.

Numerical indices of the ROC curves are frequently used to summarize the curves. These summary measures are used as the basis for comparing ROC curves. The Area Under the ROC curve (AUC) is the most widely used summary measure. A perfect diagnostic test with the perfect ROC curve has the value AUC=1.0, while an uninformative test has AUC=0.5. The statistical test proposed by DeLong et al. gives the opportunity to assess with a p-value against the null hypothesis; hence a p-value of close to zero is evidence of a classification which is significantly better than random (Biometrics; 1989; 44(3); 837-45).

Using these notions of ROC AUC and its comparisons Table 2 demonstrates ROC AUCs of two particular sets of biomarkers for predicting the probability of weight loss resistance versus weight loss sensitivity. As outlined above, the pan-Europen weight management study Diogenes (Larsen et al., as above)—in which approximately 500 subject plasma samples were available prior to the Low Calorie Diet (LCD) for measurement of 1240 proteins—was used as a discovery dataset.

For a validation data set, results from 500 independent subjects from the Ottawa Hospital Weight Management Clinic using LCD for weight loss were used.

The Ottawa cohort includes 2383 overweight and obese patients of the Weight Management Clinical of The Ottawa Hospital who underwent a meal replacement program diet combined with an exercise regime to reduce weight during 6 to 12 weeks, and were monitored for up to 36 weeks, checking their weight, height, blood pressure, at every visit, and at week 1, they had a range of biochemistry measurements performed, from lipids profile to fasting insulin and blood glucose. After exclusion criteria were applied, 2032 patients remained: 1448 females and 584 males.

Further exclusion criteria were applied based on sample available for mass-spectrometry analysis at the end of the study. Following this a group 507 proteins were measured for 500 individuals at baseline and upon completion of the study.

The final 500 subjects assessed in the Ottawa cohort were as follows:

Males (151)

| | |
|---|---|
| Age | 50.48 +− 11.55 |
| BMI W 1 | 44.82 +− 7.27 |
| BMI W 8 | 39.80 +− 6.78 |
| BMI12 | 11.25 +− 2.77 |

Females (349)

| | |
|---|---|
| Age | 47.48 +− 11.85 |
| BMI W 1 | 44.75 +− 7.24 |
| BMI W 8 | 40.64 +− 6.72 |
| BMI12 | 9.19 +− 2.56 |

Table 2 demonstrates the performance of two sets of biomarkers for predicting the outcome of LCD in two cases: (i) “best case”—when coefficients we re-estimated using logistic regression and (ii) “worst case”—when the coefficient learned on Diogenes discovery data set was used. In both cases the accuracy of stratification is significantly higher than random using E. DeLong, DeLong, and Clarke-Pearson test (Biometrics 44 (3): 837-45).

Biomarker set A includes the 21 biomarkers listed in Table 1.

Biomarker set B includes the following subset of 13 biomarkers from the biomarkers listed in Table 1: vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34 and interleukin-17 receptor C.

TABLE 2
ODR versus ODS prediction results in ROC AUC terms

| | ROC AUC | p-value |
|---|---|---|
| Discovery data set (Diogenes) - Biomarker Set A | 0.79 | 0.00 |
| Validation data set (Ottawa) - Best case | 0.92 | 0.00 |
| Biomarker Set A (using discovery platform for | | |
| measures) | | |
| Validation data set (Ottawa) - Worst case | 0.63 | 0.01 |
| Biomarker Set A (using discovery platform for | | |
| measures) | | |
| Discovery data set (Diogenes) - Biomarker Set B | 0.63 | 0.00 |
| Validation data set (Ottawa) - Best case - | 0.70 | 0.00 |
| Biomarker Set B (using discovery platform for | | |
| measures) | | |
| Validation data set (Ottawa) - Worst case - | 0.54 | 0.06 |
| Biomarker Set B (using discovery platform for | | |
| measures) | | |

The P-value is for the null hypothesis ‘the classification is as good as random’ hence low p-values (rounded to two decimal places) indicate successful classification

1. A method for predicting the degree of weight loss attainable by applying one or more dietary interventions to a subject, the method comprising:
determining the level of one or more biomarkers in one or more samples obtained from the subject, wherein the one or more biomarkers include vitamin K-dependent protein C.

2. The method according to claim 1, wherein the method further comprises determining the level of one or more biomarkers selected from the group consisting of coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

3. The method according to claim 1, wherein the method further comprises determining the level of each of coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples.

4. The method according to claim 1, wherein levels of each of vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 are determined, and increased levels of vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; protein Z-dependent protease inhibitor; serum paraoxonase/arylesterase 1; complement factor D; carbonic anhydrase 6; and angiopoietin-1 and decreased levels of leukocyte immunoglobulin-like receptor subfamily B member 2; X-ray repair cross-complementing protein 6; caspase-2; interleukin-34; interleukin-17 receptor C; plasminogen; leptin and macrophage metalloelastase in the sample is indicative of a greater degree of weight loss in the subject.

5. The method according to claim 1, wherein the one or more samples are derived from blood.

6. The method according to claim 1, wherein the dietary intervention is a low calorie diet.

7. The method according to claim 6, wherein the low calorie diet comprises a calorie intake of about 600 to about 1200 kcal/day.

8. The method according to claim 6, wherein the low calorie diet comprises administration of at least one diet product.

9. The method of according to claim 6, wherein the low calorie diet has a duration of 6 to 12 weeks.

10. The method according to claim 1, wherein the method further comprises combining the level of the one or more biomarkers with one or more anthropometric measures and/or lifestyle characteristics of the subject.

11. The method according to claim 10, wherein the anthropometric measure is selected from the group consisting of gender, weight, height, age and body mass index, and wherein the lifestyle characteristic is whether the subject is a smoker or a non-smoker.

12. The method according to claim 1, wherein the degree of weight loss is represented by the body mass index that a subject is predicted to attain by applying a dietary intervention.

13. (canceled)

14. A method for predicting the body mass index that a subject would be expected to attain from a dietary intervention (BMI2), wherein the method comprises:
a. determining the level of vitamin K-dependent protein C; coagulation factor XIII A chain; pigment epithelium-derived factor; serum amyloid P-component; neuroligin-4, X-linked; CD226 antigen; metalloproteinase inhibitor 3; eukaryotic translation initiation factor 4E-binding protein 2; leukocyte immunoglobulin-like receptor subfamily B member 2; X-ray repair cross-complementing protein 6; caspase-2; interleukin-34; interleukin-17 receptor C; protein Z-dependent protease inhibitor; serum paraoxonase/arylesterase 1; plasminogen; complement factor D; leptin; carbonic anhydrase 6; macrophage metalloelastase and angiopoietin-1 in one or more samples obtained from the subject; and
b. predicting BMI2 using formula (1):
BMI2=c1*BMI1+c2*age−c3*Vitamin K-dependent protein C−c4*Coagulation factor XIII A chain−c5*Pigment epithelium-derived factor−c6*Serum amyloid P-component−c7*Neuroligin-4,X-linked−c8*CD226 antigen−c9*Metalloproteinase inhibitor 3−c10*Eukaryotic translation initiation factor 4E-binding protein 2+c11*Leukocyte immunoglobulin-like receptor subfamily B member 2+c12*X-ray repair cross-complementing protein 6+c13*Caspase-2+c14*Interleukin-34+c15*Interleukin-17 receptor C−c16*Protein Z-dependent protease inhibitor−c17*Serum paraoxonase/arylesterase 1+c18*Plasminogen−c19*Complement factor D+c20*Leptin−c21*Carbonic anhydrase 6+c22*Macrophage metalloelastase−c23*Angiopoietin-1; (1)
wherein BMI1 is the subject's body mass index before the dietary intervention and BMI2 is the subject's predicted body mass index after the dietary intervention; and
wherein c1 to c23 are positive integers.

15. A method for selecting a modification of lifestyle of a subject, the method comprising:
a. performing a method for predicting the degree of weight loss attainable by applying one or more dietary interventions to a subject, the method comprising determining the level of one or more biomarkers in one or more samples obtained from the subject, wherein the one or more biomarkers include vitamin K-dependent protein C; and
b. selecting a suitable modification in lifestyle based upon the degree of weight loss predicted in step (a).

16. The method according to claim 15, wherein the modification of lifestyle in the subject comprises a dietary intervention.

17-26. (canceled)
